(19) United States
(12) Patent Application Publication
(10) Pub. No.: US 2026/0098289 A1
(43) Pub. Date: Apr. 9, 2026

(54) RESTRICTION ENDONUCLEASE-MEDIATED DNA STRAND DISPLACEMENT CIRCUIT

(71) Applicant: University of Washington, Seattle, WA (US)

(72) Inventors: Nuttada Panpradist, Seattle, WA (US); Jeffrey Matthew Nivala, Seattle, WA (US); Georg Seelig, Seattle, WA (US); Megan van Meurs, Seattle, WA (US)

(21) Appl. No.: 19/352,085

(22) Filed: Oct. 7, 2025

Related U.S. Application Data
(60) Provisional application No. 63/704,719, filed on Oct. 8, 2024.

(51) Int. Cl. C12Q 1/6818

(52) U.S. Cl. CPC C12Q 1/6818

(57) ABSTRACT

Nucleic acid strand displacement systems and methods for detecting presence or absence of cleavage of an endonuclease cleavage site. In an example, nucleic acid strand displacement systems comprise a nucleic acid molecule comprising: a toehold region; an endonuclease recognition cleavage site; and a toehold blocker region configured to couple to the toehold region, wherein the toehold blocker region is further configured to release from the toehold region when a cleavage occurs at the endonuclease recognition cleavage site. In an example, the system comprises a signal nucleic acid molecule configured to couple to a signal complementary region of the nucleic acid molecule. In an example, the signal nucleic acid molecule further comprises a fluorophore tag configured to generate detectable signal. In an example, the nucleic acid molecule further comprises a quencher tag configured to quench the detectable signal when the nucleic acid molecule is coupled to the signal nucleic acid molecule.

### CROSS-REFERENCE(S) TO RELATED APPLICATION(S)

This application claims the benefit and priority of U.S. Provisional Application No. 63/704,719, filed on Oct. 8, 2024, the entire disclosure of which is disclosed herein in its entirety.

### STATEMENT REGARDING SEQUENCE LISTING

The Sequence Listing XML associated with this application is provided in XML format and is hereby incorporated by reference into the specification. The name of the XML file containing the sequence listing is 3915-P1367US.UW_Sequence_Listing.xml. The XML file is 83,099 bytes; was created on Oct. 2, 2025; and is being submitted electronically via Patent Center with the filing of the specification.

### BACKGROUND

Sepsis is a life-threatening medical condition caused by the presence of harmful microorganisms in the blood, which results in dysregulated changes in physiology and biochemistry that can lead to organ damage, shock, and death. Sepsis is the final pathway toward death for many severe infectious diseases and can result from noncommunicable diseases and injuries as well. The World Health Organization estimates that 20% of all global deaths annually are due to sepsis, with 49 million cases of sepsis and 11 million sepsis-related deaths. Their 2020 report found an inverse relationship between income and sepsis incidence and mortality and highlighted the increased risks for those in lower and middle-income countries such as those in Sub-Saharan Africa where 85% of sepsis deaths occur. It is further estimated that at least 10% of maternal deaths and 26% of neonatal deaths are caused by sepsis. Death can occur in fewer than 12 hours, and thus rapid identification of the cause of the infection is crucial. Often patients must be treated with broad-spectrum antibiotics since there is no time to determine the specific cause, thus increasing microbial resistance. Additionally, past work suggests that only 15-50% of patients with sepsis are correctly coded, making sepsis care and research excessively difficult.

The average cost of treating a patient with sepsis is $32,000. At that cost, the global annual economic burden of sepsis healthcare would be 1.6 trillion dollars. In the United States, these per capita costs ranged from $39,000 in cases without organ dysfunction, to $69,000 in severe cases of septic shock. Overall, sepsis costs the US more than 24 billion dollars per year, accounting for over 6% of total healthcare expenditures, and is ranked in the top 4 most costly conditions in the US for all payer groups (Medicare, Medicaid, private insurance, and uninsured).

Survivors of sepsis are likely to need additional care facilities, suffer loss of work and bodily function, and need continued health care, costing the US an additional 2 billion dollars annually. Survivors of sepsis also often face a wide array of serious long-term health conditions due to the damage caused during the septic reaction, cognitive impairment, mental health struggles, loss of independence, and increased post-discharge mortality rates. Among adult survivors of the disease. 1 in 3 will die within a year.

Blood culturing has been the gold standard sepsis diagnostic in the industry; however, such tests can take over 72 hours, by which time the patient may likely have already passed away. With each hour of delay a patient's survival chances decrease by 7.6%. PCR is now favored in many settings, as it can produce results in as little as an hour, and is potentially more accurate than conventional methods. It is, however, significantly more expensive and energy intensive.

Multiplex PCR sepsis diagnostic tests require customized equipment that costs $35,500, and each multiplexed test panel costs >$200/test. For smaller or more rural clinics without funding or reliable access to electricity. PCR simply is not accessible. This causes the overuse of antibiotic and antimicrobial treatments, and increases bacterial resistance and patient mortality.

Although there has been research into diagnostic methods which can affordably, rapidly, and accurately detect sepsis causing pathogens, none have found full success yet. According to the World Health Organization's 2020 global report on the epidemiology and burden of sepsis, “The ideal diagnostic test for infections that cause sepsis would: 1) rapidly identify pathogens broadly (bacteria, virus, parasite, fungi): 2) be highly sensitive and specific so as to guide antimicrobial therapy, limit antibiotic overuse, and inhibit AMR development: 3) use readily available clinical samples (for example, whole blood) that do not require processing or culture: 4) allow detection of multiple pathogens simultaneously: 5) detect drug susceptibility and resistance: 6) be simple to use with minimum training required; and 7) be relatively low cost. However, many existing diagnostics, are considered too costly and technology-dependent to be realistic in LMICs”.

None of the presently available isothermal detection methods meet more than a few of these criteria. Examples of PCR-based sepsis tests cost too much money due to the expensive equipment and electricity. Loop mediated isothermal amplification (LAMP) and recombinase polymerase amplification (RPA) still require the purchasing and storage of expensive enzymes, and LAMP cannot easily be multiplexed. Fluorescence readings also cannot be multiplexed above 3 probes at once, and still require a reduced blood culturing period. Nanopore detection combined with blood culturing allows multiplexing, but it still takes 7-9 hours, which may lead to treatment that comes too late.

Accordingly, there is an unmet need for an accurate, inexpensive, quick test for sepsis deployable in low-resource settings.

### SUMMARY

The present disclosure provides nucleic acid strand displacement systems and methods for detecting the presence or absence of cleavage of an endonuclease cleavage site to address these and related challenges.

In embodiments and as described further herein, the nucleic acid strand displacement system comprises a restriction endonuclease enzyme input. In embodiments, the toehold regions are concealed and blocked by a strand that the restriction enzyme can cleave. Once cleaved, the toehold region is exposed, allowing an invading strand to hybridize and initiate the DSD cascade. As provided further herein, the systems of the present disclosure reach a 99% fluorescence yield or greater in 25 minutes with only 19% noise.

Accordingly, in an aspect, the present disclosure provides a nucleic acid strand displacement system. In an embodiment, the nucleic acid strand displacement system comprises a nucleic acid molecule comprising: a toehold region; an endonuclease recognition cleavage site; and a toehold blocker region configured to couple to the toehold region, wherein the toehold blocker region is further configured to release from the toehold region when a cleavage occurs at the endonuclease recognition cleavage site.

In another aspect, the present disclosure provides a method for detecting the presence or absence of cleavage of an endonuclease cleavage site. In an embodiment, the method comprises adding a bacteria sample to a mixture comprising: a nucleic acid molecule, wherein the nucleic acid molecule comprises: a toehold region; an endonuclease recognition cleavage site; and a toehold blocker region configured to couple to the toehold region, wherein the toehold blocker region is further configured to release from the toehold region when a restriction endonuclease cleaves the endonuclease recognition cleavage site; incubating the mixture under conditions and for a time sufficient to cleave the endonuclease recognition cleavage site in the presence of the restriction endonuclease; and detecting, in the mixture, the presence or absence of detectable signal associated with cleavage of the endonuclease recognition cleavage site.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the present disclosure will become more readily appreciated as the same become better understood by reference to the following detailed description, when taken in conjunction with the accompanying drawings, wherein:

FIG. 1 is a schematic diagram of nucleic acid strand displacement system, according to embodiments of the present disclosure:

FIGS. 2A-2E schematically illustrate a protocol for using nucleic acid strand displacement system, according to embodiments of the present disclosure:

FIG. 3 provides an illustration of a nucleic acid strand displacement system according to embodiments of the present disclosure, and associated predicted secondary structures, melting temperatures, and Gibb's free energy values:

FIG. 4 provides an illustration of a nucleic acid strand displacement system, according to embodiments of the present disclosure, and associated predicted secondary structure, melting temperature, and Gibb's free energy value;

FIGS. 5A-5F illustrate optimization of restriction endonuclease mediated DNA strand displacement (resDSD), according to embodiments of the present disclosure, where real-time data of FAM signal across a 1-h incubation at 37° C. when the invading strand was used (5A) at 1.5 μM (5B) 2 μM (5C) 2.5 μM, according to embodiments of the present disclosure, and lines are averages with the upper and lower bound of averages±SE (n=4). FAM signal measured at 25 minutes when the invading strand was used at (5D) at 1.5 μM (5E) 2 μM (5F) 2.5 μM, according to embodiments of the present disclosure, where bars are averages with SE (n=4). * p-value <0.05 (T-test, two-tailed, unequal variance with Holm Bonferroni corrections for multiple comparisons):

FIG. 6 provides an illustration of a nucleic acid strand displacement system, according to embodiments of the present disclosure, and associated predicted secondary structure, melting temperature, and Gibb's free energy value:

FIG. 7 illustrates real-time data of FAM signal across a 25 min incubation at 37° C. when invading strand was used at 2.5 uM, according to embodiments of the present disclosure:

FIG. 8 illustrates real-time data of FAM signal for a nucleic acid strand displacement system across a 25 min incubation at 37° C. when invading strand was used at 2.8 uM, according to embodiments of the present disclosure:

FIG. 9 illustrates real-time data of FAM signal for nucleic acid strand displacement system, contaminated with E. coli and human genomic DNA, across a 35 min incubation at 37° C. when the invading strand was used at 2.8 uM, according to embodiments of the present disclosure; and

FIG. 10 provides a t-test, two-tailed, unequal variance, Holm-Bonferroni correction for the contamination experiment, according to embodiments of the present disclosure.

### DETAILED DESCRIPTION

In various aspects, the present disclosure provides nucleic acid strand displacement systems and methods for detecting the presence or absence of cleavage of an endonuclease cleavage site.

In an aspect, the present disclosure provides nucleic acid strand displacement system. In an embodiment, the nucleic acid strand displacement system comprises a nucleic acid molecule comprising: a toehold region: an endonuclease recognition cleavage site; and a toehold blocker region configured to couple to the toehold region, wherein the toehold blocker region is further configured to release from the toehold region when a cleavage occurs at the endonuclease recognition cleavage site.

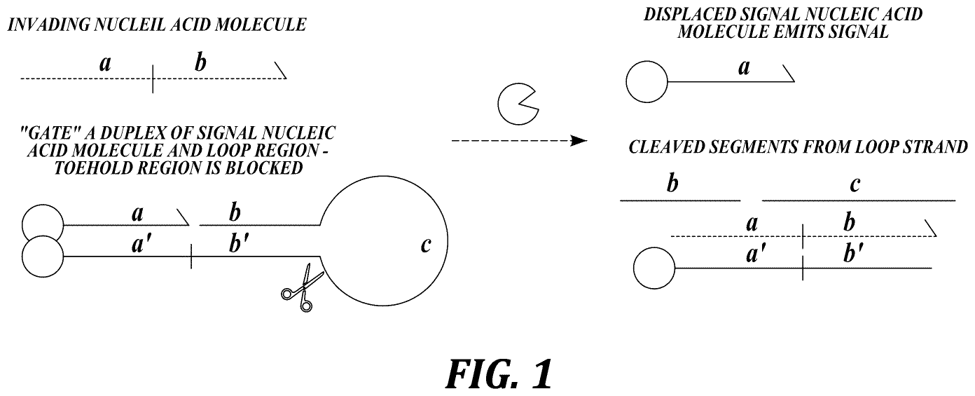

An embodiment of a nucleic acid strand displacement system and its operation is illustrated in FIG. 1. As shown, the nucleic acid strand displacement system comprising a nucleic acid molecule comprising: a toehold region (b′): an endonuclease recognition cleavage site; and a toehold blocker region (b) configured to couple to the toehold region, wherein the toehold blocker region is further configured to release from the toehold region when a cleavage occurs at the endonuclease recognition cleavage site. In an embodiment, the endonuclease recognition cleavage site is formed by the coupling of the toehold region (b) to the toehold blocker region (b).

In the illustrated embodiment, the nucleic acid strand displacement system is also shown to include a signal nucleic acid molecule (a) configured to couple to a signal complementary region (a′) of the nucleic acid molecule. As also shown, the signal nucleic acid molecule (a) further comprises a fluorophore tag configured to generate a detectable signal. As shown, the nucleic acid molecule further comprises a quencher tag configured to quench the detectable signal when the nucleic acid molecule is coupled to the signal nucleic acid molecule.

The nucleic acid strand displacement system is shown to comprise an invading nucleic acid molecule (a/b) configured to couple to the toehold region (b) and the signal complementary region (a′), as well as a loop region (c) connecting the toehold region (b′) and the toehold blocker region (b).

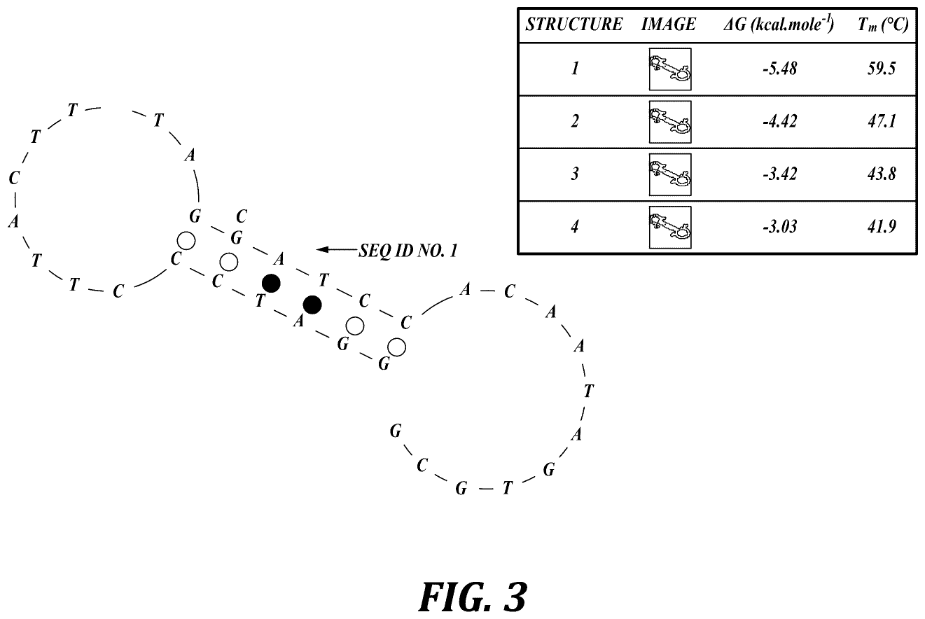

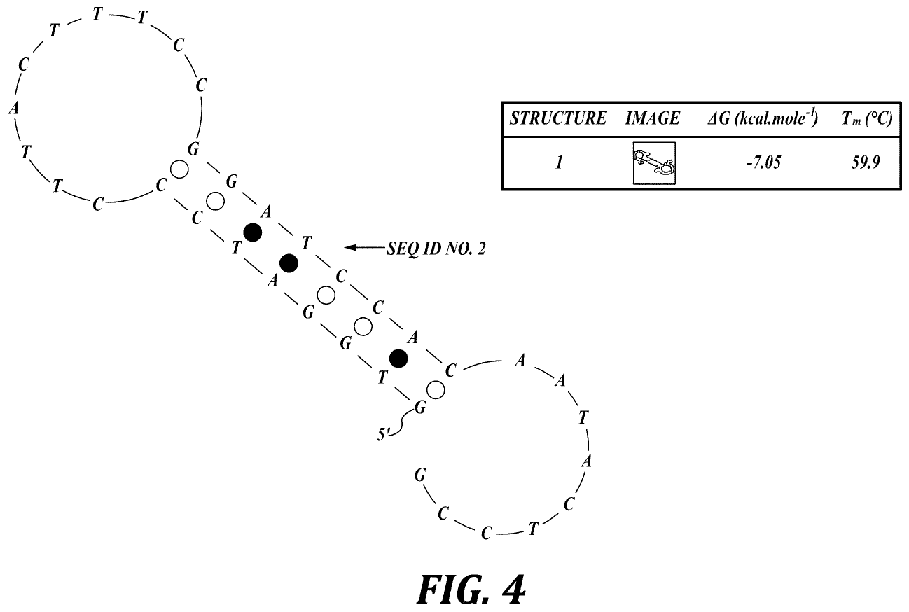

Other examples of nucleic acid strand displacement systems according to embodiments of the present disclosure are illustrated in FIGS. 3 (SEQ ID NO. 1), 4 (SEQ ID NO. 2), and 6 (SEQ ID NO. 3).

In an embodiment, the nucleic acid strand displacement systems comprise a nucleic acid molecule according to one or more of SEQ ID NOS. 4, 10-12, 15-20, 23, 26, 29-31, 34-36, 39, 42, 47, 50, 55-57, 60, 71, 74, 78-82, 87, and 90. In an embodiment, the nucleic acid strand displacement systems comprise a nucleic acid molecule at least 75% identical a nucleic acid molecule according to one or more of SEQ ID NOS. 4, 10-12, 15-20, 23, 26, 29-31, 34-36, 39, 42, 47, 50, 55-57, 60, 71, 74, 78-82, 87, and 90.

In an embodiment, the nucleic acid strand displacement systems comprise an invading nucleic acid molecule according to one or more of SEQ ID NOS. 8, 14, 21, 24, 27, 32, 37, 40, 45, 53, 58, 72, 76, 85, and 88. In an embodiment, the nucleic acid strand displacement systems comprise an invading nucleic acid molecule at least 75% identical an invading nucleic acid molecule according to one or more of SEQ ID NOS. 8, 14, 21, 24, 27, 32, 37, 40, 45, 53, 58, 72, 76, 85, and 88.

In an embodiment, the nucleic acid strand displacement systems comprise a signal nucleic acid molecule according to one or more of SEQ ID NOS. 9, 13, 22, 25, 28, 33, 38, 41, 46, 49, 51, 54, 59, 73, 75, 77, 83, 86, and 89. In an embodiment, the nucleic acid strand displacement systems comprise a signal nucleic acid molecule an invading nucleic acid molecule a signal nucleic acid molecule according to one or more of SEQ ID NOS. 9, 13, 22, 25, 28, 33, 38, 41, 46, 49, 51, 54, 59, 73, 75, 77, 83, 86, and 89.

As discussed further herein, in embodiments, the nucleic acid strand displacement systems can comprise a number of nucleic acid molecules, such nucleic acid molecules comprising an endonuclease cleavage site, an invading nucleic acid molecule, a signal nucleic acid molecule, and the like, configured to operate in the presence of an endonuclease to provide a detectable signal. Accordingly, in embodiments, the nucleic acid strand displacement system comprise a plurality of nucleic acid molecules according to embodiments of the present disclosure.

In an embodiment, the nucleic acid strand displacement systems comprise nucleic acid molecules according to one or more of SEQ ID NOS. 4-7.

In an embodiment, the nucleic acid strand displacement systems comprise nucleic acid molecules according to one or more of SEQ ID NOS. 6-10.

In an embodiment, the nucleic acid strand displacement systems comprise nucleic acid molecules according to one or more of SEQ ID NOS. 11, 15, and 16.

In an embodiment, the nucleic acid strand displacement systems comprise nucleic acid molecules according to one or more of SEQ ID NOS. 17-22.

In an embodiment, the nucleic acid strand displacement systems comprise nucleic acid molecules according to one or more of SEQ ID NOS. 23-30.

In an embodiment, the nucleic acid strand displacement systems comprise nucleic acid molecules according to one or more of SEQ ID NOS. 31-35.

In an embodiment, the nucleic acid strand displacement systems comprise nucleic acid molecules according to one or more of SEQ ID NOS. 36-39.

In an embodiment, the nucleic acid strand displacement systems comprise nucleic acid molecules according to one or more of SEQ ID NOS. 40-46.

In an embodiment, the nucleic acid strand displacement systems comprise nucleic acid molecules according to one or more of SEQ ID NOS. 47-53.

In an embodiment, the nucleic acid strand displacement systems comprise nucleic acid molecules according to one or more of SEQ ID NOS. 55-57.

In an embodiment, the nucleic acid strand displacement systems comprise nucleic acid molecules according to one or more of SEQ ID NOS. 58-71.

In an embodiment, the nucleic acid strand displacement systems comprise nucleic acid molecules according to one or more of SEQ ID NOS. 72-75.

In an embodiment, the nucleic acid strand displacement systems comprise nucleic acid molecules according to one or more of SEQ ID NOS. 76-80.

In an embodiment, the nucleic acid strand displacement systems comprise nucleic acid molecules according to one or more of SEQ ID NOS. 81-84.

In an embodiment, the nucleic acid strand displacement systems comprise nucleic acid molecules according to one or more of SEQ ID NOS. 85-90.

As used herein, “at least 75% identical” or “having at least 75% sequence identity” means that the nucleic acid molecule differs in its full length nucleic acid sequence by 25% or less (including any nucleic acid residue substitutions, deletions, additions, or insertions) relative to a reference sequence.

In an embodiment, the toehold blocker region is at least partially complementary to the toehold region.

In an embodiment, the nucleic acid strand displacement system comprises a restriction endonuclease configured to cleave the endonuclease recognition cleavage site.

In an embodiment, the nucleic acid strand displacement system comprises a signal nucleic acid molecule configured to couple to a signal complementary region of the nucleic acid molecule. In an embodiment, the signal nucleic acid molecule further comprises a fluorophore tag configured to generate a detectable signal. In an embodiment, the nucleic acid molecule further comprises a quencher tag configured to quench the detectable signal when the nucleic acid molecule is coupled to the signal nucleic acid molecule. In an embodiment, the nucleic acid strand displacement system comprises an invading nucleic acid molecule configured to couple to the toehold region and the signal complementary region.

In an embodiment, the nucleic acid molecule further comprises a loop region connecting the toehold region and the toehold blocker region. In an embodiment, the loop region comprises the endonuclease recognition cleavage site. In an embodiment, by having the loop region connect the toehold region and the toehold blocker region, the system is more sensitive. In this regard, when the loop region is cleaved by an appropriate endonuclease the nucleic acid molecule is no longer unimolecular and the melting temperature of the various remaining portions decreases significantly. Accordingly, in the presence of an appropriate endonuclease, a large difference in signal is provided, such as at lower temperatures.

In an embodiment, the nucleic acid strand displacement system is configured to detect or generate a detectable signal based on the presence or absence of several endonucleases. In this regard, in an embodiment, the nucleic acid molecule is a first nucleic acid molecule, the toehold region is a first toehold region, the endonuclease recognition cleavage site is a first endonuclease recognition site, the toehold blocker region is a first blocker region. In an embodiment, the nucleic acid strand displacement system further comprises a second nucleic acid molecule comprising: a second toehold region: a second endonuclease recognition cleavage site; and a second toehold blocker region configured to couple to the second toehold region, wherein the second toehold blocker region is further configured to release from the second toehold region when a second cleavage occurs at the second endonuclease recognition cleavage site.

In an embodiment, the first endonuclease recognition cleavage site is different than the second endonuclease recognition cleavage site.

In an embodiment, the first endonuclease recognition cleavage site is configured to be cleaved by a first restriction endonuclease, and wherein the second endonuclease recognition cleavage site is configured to be cleaved by a second restriction endonuclease different than the first restriction endonuclease.

In an embodiment, the nucleic acid strand displacement system further comprises: a first signal complementary region configured to couple to the first signal nucleic acid molecule, wherein the first signal nucleic acid molecule comprises a first fluorophore tag configured to generate a detectable signal: a first invading nucleic acid molecule configured to couple to the first toehold region and the first signal complementary region: a second signal complementary region of the second nucleic acid molecule: a second signal nucleic acid molecule configured to couple to the second signal complementary region: a second invading nucleic acid molecule configured to couple to the second toehold region and the second signal complementary region: a second fluorophore tag coupled to the second signal nucleic acid molecule.

In an embodiment, the first fluorophore tag and the second fluorophore tag are configured to generate different detectable signals. In this regard, the nucleic acid strand displacement system is configured to generate two different detectable signals based on the presence of different endonucleases.

In an embodiment, the nucleic acid strand displacement system is a sepsis diagnostic configured to detect the presence of target bacteria in under an hour using only a heat block, a Nanopore machine, and a consumable test costing <$1, which is shelf stable and easy to transport.

As described and demonstrated further herein, in an embodiment, in operation of the nucleic acid strand displacement system, such as according to operation of the methods of the present disclosure, a DNA input “invading” strand invades a duplex DNA substrate, replacing the previous incumbent strand through branch migration to reveal a fluorescence molecule. In the process of DNA strand displacement (DSD), an invading strand replaces a placeholder strand bound to a complementary strand. For diagnostics, a detectable molecule such as a magnetic bead or fluorophore can then be released from a blocking or quenching molecule of one of the originally bound strands. In toehold-mediated DSD, a toehold sequence is exposed, either by design or by cleavage of the DNA, and this initiates the displacement reaction. The more thermodynamically and kinetically favorable strand will “invade” and replace the less favorable placeholder strand. DSD methodology relies on DNA thermodynamics and kinetics predictions in order to determine the sequences that can be reliably used.

According to embodiments of the present disclosure, the nucleic acid strand displacement system employs a restriction endonuclease enzyme input. In embodiment, the toehold regions are concealed and blocked by a strand that the restriction enzyme can cleave. Once cleaved, the toehold region is exposed, allowing an invading strand to hybridize and initiate the DSD cascade.

Combined with corresponding nucleic acid strand displacement system, variously referred to herein as “resDSD circuits”, the ensuing reaction allows for rapid detection of the pathogens. DNA circuits can be dried into a pellet to be used in the consumable test.

In embodiment, the nucleic acid strand displacement systems are rapid, affordable, use whole blood, require minimal training, and test for a range of the most common bacterial strains simultaneously at a high level of specificity and sensitivity.

In embodiment, the nucleic acid strand displacement system is configured to test for a range of common bacterial strains simultaneously at a high level of specificity and sensitivity.

In embodiment, the melting temperatures of the loop, barcode, invading strand are at least 5-10 C higher than 37 C. In this regard, the temperature of the reaction, and the strands naturally bind under those conditions. In embodiments, the section of the hairpin left bound to the toehold after being cut by the restriction enzyme conversely has a melting temperature much lower than that of the reaction, such that it unbinds (e.g., melts) and exposes the toehold region.

In order to ensure that displacement will occur, the invading strand melting temperature, in embodiments, is significantly larger than that of the barcode strand, thus making the invading strand more thermodynamically preferential. In an embodiment, the ΔG of the loop/hairpin should is as small as possible, since the smaller the Gibb's free energy, the more energetically preferential it is. A strong loop provides that the circuit does not unbind in the absence of the restriction endonuclease.

In an embodiment, the length of each strand is less than 50 bp, as above this length the price to order the DNA increases drastically due to the need for purification.

In an embodiment, the fluorophore chosen to attach to the barcode strand is compatible with plate reader analysis. In an embodiment, the barcode strand is long enough to be compatible with future nanopore sequencing (e.g., at least 20 bp).

In an embodiment, the nucleic acid strand displacement system comprises a 2:1 ratio of loop to barcode strand. This provides that all or at least most or at least some of barcode is quenched. In an embodiment, there is an excess of invading strand, ensuring that the invading strand will find a toehold via random kinetics. In an embodiment, the enzyme chosen to test the circuit must be functional at 37 C and should be easily commercially available. In an embodiment, the enzyme has a known and specific targeted cutsite.

In another aspect, the present disclosure kits comprising nucleic acid strand displacement systems according to any embodiment of the present disclosure and instructional materials on how to use the nucleic acid strand displacement systems, such as to perform one or more methods of the present disclosure as described further herein.

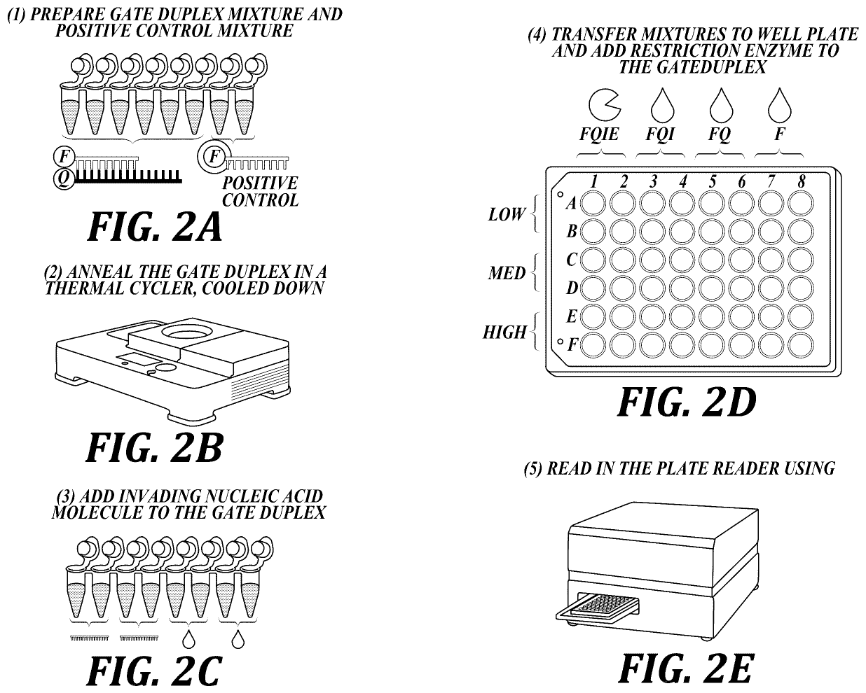

In another aspect, the present disclosure provides a method for detecting the presence or absence of cleavage of an endonuclease cleavage site. In an embodiment, the method is performed using a nucleic acid strand displacement system according to any embodiment of the present disclosure. In an embodiment, the method is schematically illustrated in FIGS. 2A-2E.

In an embodiment, the method comprises adding a bacteria sample to a mixture comprising: a nucleic acid molecule, wherein the nucleic acid molecule comprises: a toehold region; an endonuclease recognition cleavage site; and a toehold blocker region configured to couple to the toehold region, wherein the toehold blocker region is further configured to release from the toehold region when a restriction endonuclease cleaves the endonuclease recognition cleavage site; incubating the mixture under conditions and for a time sufficient to cleave the endonuclease recognition cleavage site in the presence of the restriction endonuclease; and detecting, in the mixture, the presence or absence of detectable signal associated with cleavage of the endonuclease recognition cleavage site.

In an embodiment, the nucleic acid molecule further comprises a loop region connecting the toehold region and the toehold blocker region, and wherein the loop region comprises the endonuclease recognition cleavage site.

In an embodiment, the mixture further comprises a signal nucleic acid molecule configured to couple to a signal complementary region of the nucleic acid molecule, and wherein the signal nucleic acid molecule further comprises a fluorophore tag configured to generate a fluorescent detectable signal.

In an embodiment, the nucleic acid molecule further comprises a quencher tag configured to quench fluorescence of the fluorophore tag.

In an embodiment, the mixture further comprises an invading nucleic acid molecule configured to couple to the toehold region and the signal complementary region.

In an embodiment, detecting comprises detecting the presence or absence of the detectable signal, wherein the presence of the first detectable signal is indicative of the presence of the restriction endonuclease in the mixture.

Unless stated otherwise, experimental hypotheses or forward-looking models and statements are not intended to be binding on the applicant or exhaustive of the range of possible experimental hypotheses or forward-looking models and statements, but rather are intended to be illustrative, non-limiting examples for aiding those in the art in the understanding and practice of elements of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense: that is to say, in the sense of “including, but not limited to”.

Unless the context clearly requires otherwise, the phrase “consisting essentially of” limits the scope of a claim to the specified materials or steps and those that do not materially affect the basic and novel characteristic(s) of the claim.

Unless the context clearly requires otherwise, the phrase “consisting of” excludes any element, step, or ingredient not specified.

If an element is described or claimed herein such that it “comprises” a feature, that description or claim also includes embodiments wherein the element “consists essentially of” and embodiments wherein the element “consists of” the feature, unless something else is specifically stated to the contrary.

A nucleic acid is a polymer of monomer units or “residues”. The monomer subunits, or residues, of the nucleic acids each contain a nitrogenous base (i.e., nucleobase), a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA. The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, or a ribonucleotide (RNA) polymer. The nucleic acids can also be or comprise a PNA polymer, or a combination of any of the polymer types described herein (e.g., contain residues with different sugars).

Unless stated otherwise herein, 1-letter abbreviations for nucleic acids are consistent with the nomenclature used in the art (i.e., A, Adenine: T, Thymine: C, Cytosine; G, Guanine: U, Uracil). Unless stated otherwise herein, 1-letter abbreviations for amino acids are consistent with the nomenclature used in the art (i.e., Alanine, A; Arginine, R; Asparagine, N; Aspartic acid, D; Cysteine, C; Glutamic acid, E; Glutamine, Q; Glycine, G; Histidine H; Isoleucine, I; Leucine, L; Lysine, K; Methionine, M; Phenylalanine, F; Proline, P; Serine, S; Threonine, T; Tryptophan, W; Tyrosine, Y; Valine, V).

Unless stated otherwise herein, the terms “nucleic acid,” “amino acid,” “nucleotide,” and “peptide” are inclusive and open-ended, and do not exclude from their scope any chemically modified or post-translationally modified versions of these structures, and also do not exclude from their scope any nuclear modified versions, for example, due to the presence of one or more radioisotopes in one or more of these structures.

Unless otherwise stated or the context clearly requires otherwise, methods of the disclosure can be performed, in whole or in part, in any order of steps, including steps that are performed subsequently, in parallel, and in combination. In addition, methods can be performed, in whole or in part, by humans optionally assisted by one or more machines such as one or more computational devices or systems (e.g., computer(s)). In at least some instances, methods can be performed by one or more humans with little or no substantive assistance by one or more machines. In at least some other instances, methods can be performed by one or more humans with substantive assistance by one or more machines, and in at least some instances, one or more machines can perform methods autonomously or semi-autonomously.

As used herein, an “instructional material” includes a publication, a recording, a diagram, or any other medium of expression which can be used to communicate the usefulness of one or more elements of a kit of the disclosure for carrying out a method of the disclosure, including methods for detecting the presence or absence of cleavage of an endonuclease cleavage site as described herein. The instructional material of the kit can, for example, be affixed to a container which contains an identified compound or enzyme, such as a non-specific adenine methyltransferase. The instructional material can be shipped together with a container which contains the identified compound or enzyme. Alternatively, the instructional material can be shipped separately from the container with the intention that the instructional material and the compound or enzyme be used cooperatively.

Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that can vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the cited references and disclosure to provide yet further embodiments of the disclosure. These and other changes can be made to the disclosure in light of the detailed description.

It will be appreciated that, although specific embodiments of the disclosure have been described herein for purposes of illustration, various modifications can be made without deviating from the spirit and scope of the disclosure. Accordingly, the disclosure is not limited except as stated by the claims.

While illustrative embodiments have been illustrated and described, it will be appreciated that various changes can be made therein without departing from the spirit and scope of the disclosure.

### EXAMPLES

### Example 1: Circuit Design

The circuit comprises three strands: (i) a 5′ fluorophore-labeled incumbent strand, (ii) a 3′ Quencher-labeled strand with a complementary region to the incumbent strand, and a 5′ loop with a double-stranded region containing the BamHi restriction site, and (iii) an unlabeled invading DNA strand.

To design the resDSD circuit, an in-house MATLAB algorithm was utilized to generate random sequences of specific lengths, which were then combined with the BamHi recognition sequence.

Binding probabilities and melting temperatures were calculated using IDT's DNA Oligoanalyzer [38]. The melting temperature is calculated using nearest neighbor calculations, where the enthalpy and entropy of each dinucleotide pair are considered. The thermodynamics equation is as follows:

$${T}_{m} = \frac{Δ H}{Δ S + Rln ( C )}$$

ΔH is the sum of the enthalpy changes of all nearest-neighbor pairs.

ΔS is the sum of the entropy changes of all nearest-neighbor pairs.

R is the universal gas constant (1.987 cal/(K·mol)). C is the concentration of the oligonucleotide strands

Then the Owczarzy salt correction is applied. This considers the effect of the Na+ and Mg++ ions in the solution. Then the Gibbs free energy. ΔG=ΔH−TΔS, and the binding affinity K=e^{-ΔG/(RT)}, are used to calculate the binding probabilities between and amongst the oligos.

The randomized sequences were then optimized to minimize undesired secondary structures and ensure melting temperatures were ≥5° C. higher than the BamHi optimal temperature (37° C.). During the optimization process, the length of the bound portion of the looped strand was extended downstream of the cut site so as to increase the melt temperature of the loop without affecting the melting temperature of the incumbent strand. The GC content of the incumbent strand was optimized to maximize the difference in melting temperature between the incumbent and invading strands. This was done to minimize noise from the invading strand out competing the incumbent strand in the absence of the restriction enzyme cutting the loop and exposing the toehold.

### Example 2: Testing the Circuit

The positive control (F) had only the fluorophore-labeled incumbent strand. The other conditions contained mixtures of 1 μM fluorophore and 2 μM quencher strands which were subjected to a 2-minute incubation at 95° C., followed by a 40-minute annealing step from 95° C. to 25° C. Negative control (FQ) had fluorophore and quencher strands without any BamHi or invading strand added. To differentiate the contribution of yield from DSD and resDSD, the FQ mixture was mixed with 1.5-2.5 μM invading strands without adding BamHi (DSD control, FQI) or with 60 U BamHi enzyme (resDSD test case, FQIE). All four reaction conditions (F, FQ, FQI, and FQIE; n=4 each) were incubated at 37° C. and read every minute for one hour (Excitation/Emission: 485/528, Gain: 55). ResDSD yields were calculated from FQIE signal/F signal. Noises from DSD were calculated from the FQI signal/FQIE signal.

### Contaminating the Circuit

Contamination validation experiments were performed to determine if the circuit would continue to perform when contaminated with human genomic DNA and Escherichia coli (E. coli). The same test as before was performed with one uncontaminated control and one condition that is contaminated with 0.2 ng of human DNA and 2\hat{ }8 CFU/mL E. coli. The invading strand concentration was also increased to 2.8 μM.

### Example 3: Results

For the first design, the bound section of the hairpin was only composed of the cut site. There were also a number of predicted secondary structures which could occur whose melting temperatures were above 37 C. The barcode's melting temperature was also 41.7 C, which although higher than 37 C, is not 5 C or more higher.

For the redesign, the length of the loop strand was extended from 33 bp to 35 bp. The overlapping section of the hairpin was extended from 6 bp to 8 bp. The predicted melting temperature of the hairpin increased slightly from 59.5 C to 59.9 C. and the ΔG of the hairpin decreased from −5.48 to −7.05. There was also only one predicted secondary structure for the loop strand.

Experiments were then conducted to validate these predictions.

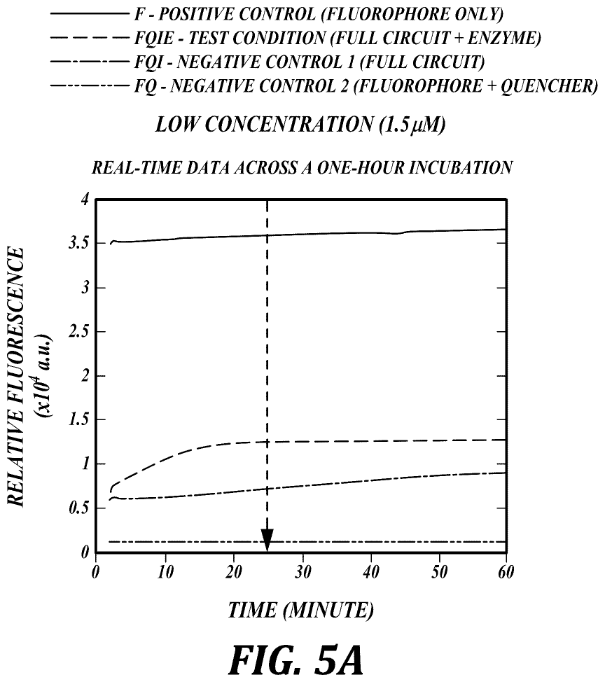

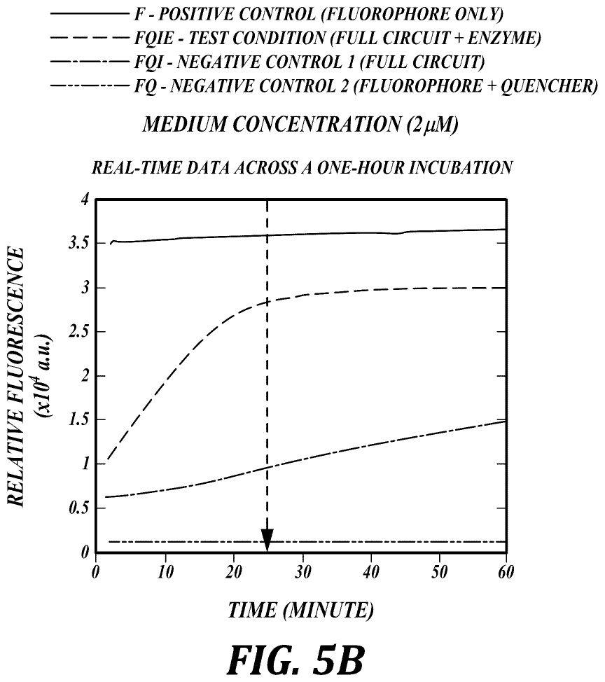

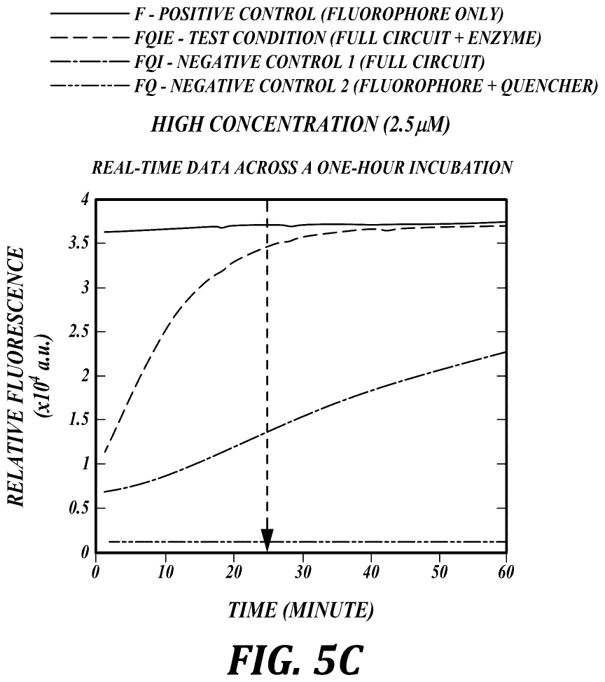

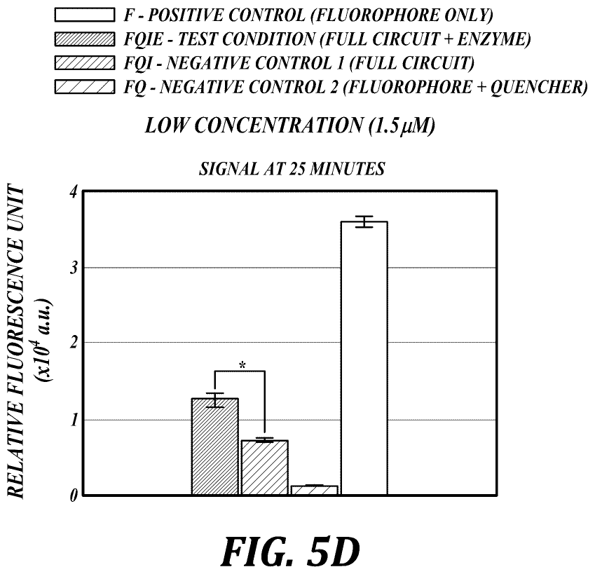

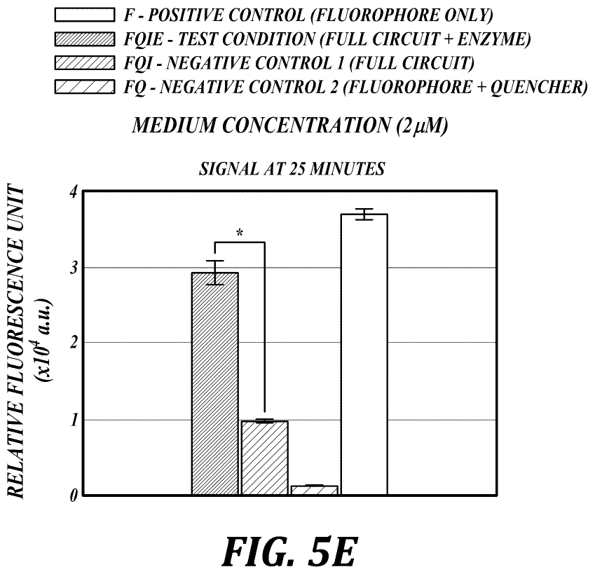

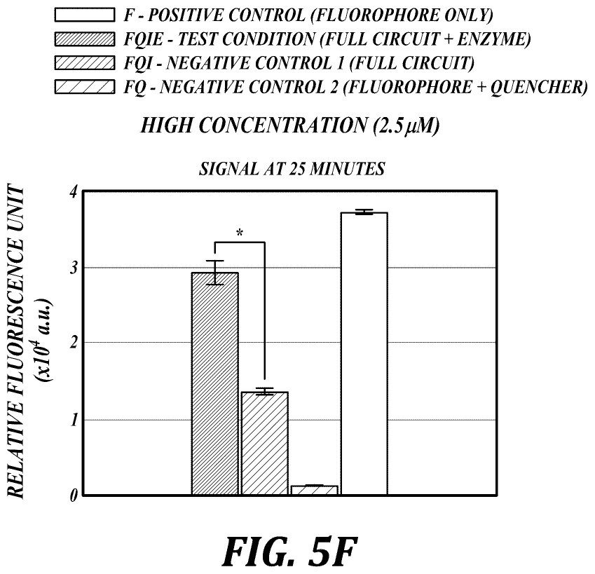

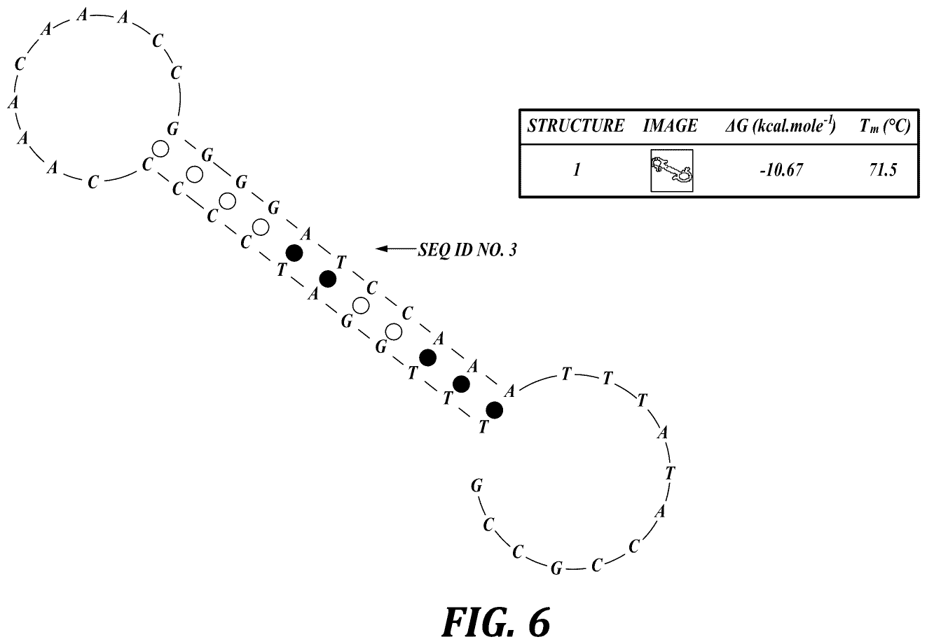

Optimization of our resDSD reaction (FIGS. 5A-5F) was performed using three invading strand concentrations. In all conditions, FQIE signals (i.e., resDSD reactions) were significantly higher than their respective FQI signals (i.e., DSD reactions); P-values (T-test, two-tailed, unequal variance, Holm-Bonferroni correction) were <0.008, <0.002, and <0.0003 for 1.5, 2, and 2.5 μM invading strand, respectively. At the highest concentration (2.5 μM) of the invading strand, we observed the highest resDSD yield of 93±0.2% (mean±SE) with 39±0.3% yield contributed from the DSD noise (FQI) after a 25-minute incubation. A longer incubation increased the yield to 99±0.04%, but increased noise to 62±0.2%. This signal-to-noise ratio pattern was also observed when the medium invading strand concentration (2 μM) was used but the total resDSD yield was dropped to 79±0.6% with 33±0.6% contributed from the DSD noise. At the low invading strand concentration (1.5 μM), we only observed 35±1% resDSD yield with 58±1% of the yield contributed from DSD noise.

Despite these relatively high levels of noise contributed by DSD, we observed the significant effect of resDSD. New designs that increase the length of the toehold blocking region will increase the stability of the circuit gate and likely improve the specificity of our resDSD assay.

For the second redesign, the length of the loop strand was extended from 35 bp to 44 bp. The overlapping section of the hairpin was extended from 8 bp to 11 bp. The predicted melting temperature of the hairpin increased significantly from 59.0 C to 71.5 C. and the ΔG of the hairpin decreased from −7.05 to −10.67. There was only one predicted secondary structure for the loop strand. Experiments were then conducted again to validate these predictions, and determine if these changes would improve the yield of the circuit whilst reducing the noise.

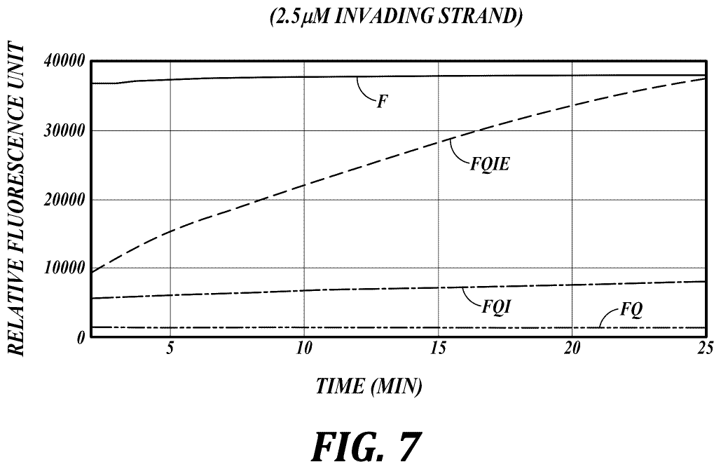

The FQIE signal (i.e., resDSD reaction) is significantly higher than the FQI signal (i.e., DSD reactions); P-value (T-test, two-tailed, unequal variance) was <9E-11 for 2.5 μM invading strand. We observed the highest resDSD yield of 99±0.1% (mean±SE) with 19±0.1% yield contributed from the DSD noise (FQI) after a 25-minute incubation. See FIG. 7.

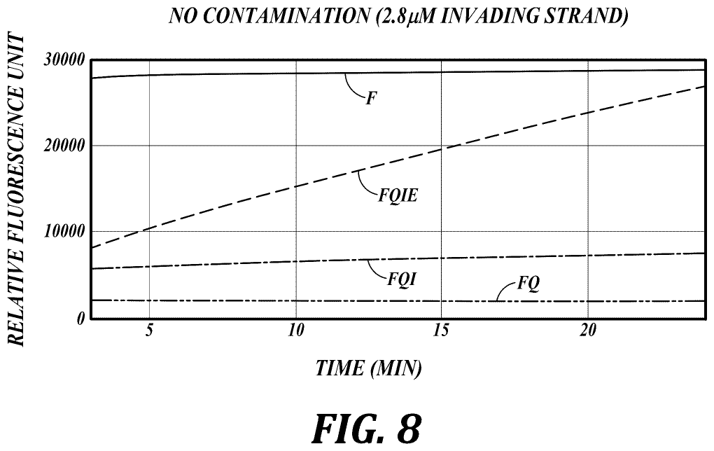

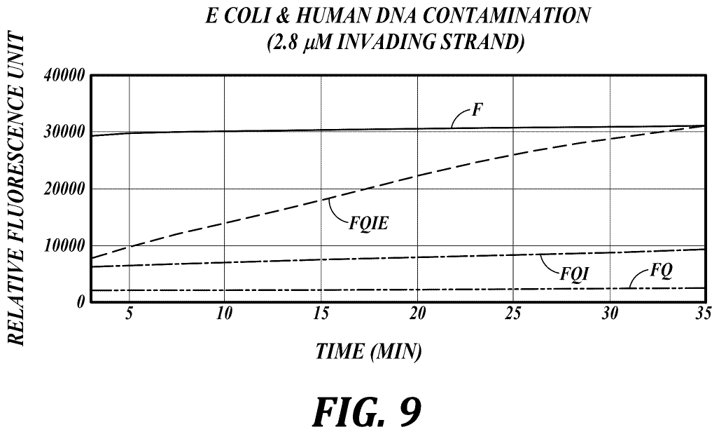

In both conditions, FQIE signals (i.e., resDSD reactions) were significantly higher than their respective FQI signals (i.e., DSD reactions); P-values (T-test, two-tailed, unequal variance, Holm-Bonferroni correction) were <0.02, and <0.004 for the non contaminated and contaminated conditions, respectively. For the non contaminated condition, we observed the highest resDSD yield of 99±0.01% (mean±SE) with 22±0.01% yield contributed from the DSD noise (FQI) after a 25-minute incubation. For the contaminated condition, we observed a slightly decreased value of 88±0.001% resDSD yield with 22±0.001% of the yield contributed from DSD noise. If the time is extended from 25 minutes to 32 minutes, the yield for the contaminated condition increases to 100±0.001% and noise actually decreases very slightly to 21±0.001%. See FIGS. 8 and 9.

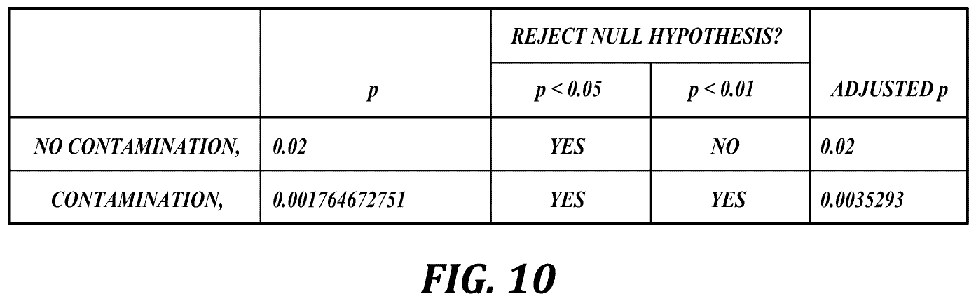

The p-values are less robust, but still statistically significant. See FIG. 10.

TABLE 1
Nucleic Acid Sequences

| SEQ ID NO. S | Sequence |
|---|---|
| SEQ ID NO. 1 | ggatccctta ctttaggatc cacaatagtg cg |
| SEQ ID NO. 2 | gtggatccct tactttccgg atccacaata ctccg |
| SEQ ID NO. 3 | tttggatccc ccaaacaaac cggggatcca aatttatacc gccg |
| SEQ ID NO. 4 | tttggatccc ccaaacaaac cggggatcca aatttatacc gccg |
| SEQ ID NO. 5 | cggcggtata aa |
| SEQ ID NO. 6 | cggcggtata aattt |
| SEQ ID NO. 7 | cggcggtata aatttggatc |
| SEQ ID NO. 8 | cggcggtata aatttggatc |
| SEQ ID NO. 9 | cggcggtata aattt |
| SEQ ID NO. 10 | tttggatccc ccaaacaaac cggggatcca aatttatacc gccg |
| SEQ ID NO. 11 | atttggatcc gcccggaaac aacccgggcg gatccaaatt tataccgccg |
| SEQ ID NO. 12 | tttggatccc ttactttccg gatccaaaga aaaaccgccg |
| SEQ ID NO. 13 | cggcggtttt tttt |
| SEQ ID NO. 14 | cggcggtttt ttttggatc |
| SEQ ID NO. 15 | atttggatcc gcccggaaac aacccgggcg gatccaaatt tataccgccg |
| SEQ ID NO. 16 | cggcggtata aatttggatc cgcccgggtt gtttccgggc ggatccaaat |
| SEQ ID NO. 17 | atttggatcc gcccggccca aacaaaccgg ccgggcggat ccaaatttat accgccg |
| SEQ ID NO. 18 | cggcggtata aatttggatc cgcccggccg gtttgtttgg gccgggcgga tccaaat |
| SEQ ID NO. 19 | tttggatccc cccccaaaca aaccgggggg gatccaaatt tataccgccg |
| SEQ ID NO. 20 | tttggatccc ccaaacaaac cggggatcca aatttatacc gccg |
| SEQ ID NO. 21 | cggcggtata aatttggatc |
| SEQ ID NO. 22 | cggcggtata aattt |
| SEQ ID NO. 23 | tttggatccc ttactttccg gatccaaaga aaaaccgccg |
| SEQ ID NO. 24 | cggcggtttt ttttggatc |
| SEQ ID NO. 25 | cggcggtttt tttt |
| SEQ ID NO. 26 | tttggatccc ttactttccg gatccaaaga aaaaccgccg |
| SEQ ID NO. 27 | cggcggtttt tctttggatc |
| SEQ ID NO. 28 | cggcggtttt tttt |
| SEQ ID NO. 29 | tttggatccc ttactttccg gatccaaaga aaaaccgccg |
| SEQ ID NO. 30 | cggcggtttt ttttggatc cggaaagtaa gggatccaaa |
| SEQ ID NO. 31 | gtggatccct tactttccgg atccacaaac cgccg |
| SEQ ID NO. 32 | cggcggtttg tggatc |
| SEQ ID NO. 33 | cggcggtttg t |
| SEQ ID NO. 34 | gtggatccct tactttccgg atccacaaac cgccg |
| SEQ ID NO. 35 | cggcggtttg tggatccgga aagtaaggga tccac |
| SEQ ID NO. 36 | gtggatccct tactttccgg atccacaata ctccg |
| SEQ ID NO. 37 | cggagtattg tggatc |
| SEQ ID NO. 38 | cggagtattg t |
| SEQ ID NO. 39 | gtggatccct tactttccgg atccacaata ctccg |
| SEQ ID NO. 40 | gtcccctccc cctcccgtcc ctccggatc |
| SEQ ID NO. 41 | aaaataataa aaaaatatgt cccctccccc tcccgtccct cc |
| SEQ ID NO. 42 | tggatccctt tcttttcgga tccggaggga cgggaggggg aggggacata tttttttatt atttt |
| SEQ ID NO. 43 | gcctccccct cccgtccc |
| SEQ ID NO. 44 | ggagggacgg gagggggagg ggacatattt ttttattatt tt |
| SEQ ID NO. 45 | gatccggagg gacgggaggg ggaggggac |
| SEQ ID NO. 46 | ggagggacgg gagggggagg ggacatattt ttttattatt tt |
| SEQ ID NO. 47 | tggatccctt tcttttcgga tccgggacgg gagggggagg catatttttt tattatttt |
| SEQ ID NO. 48 | gcctccccct cccgtcccgg atc |
| SEQ ID NO. 49 | aaaataataa aaaaatatgc ctccccctcc cgtccc |
| SEQ ID NO. 50 | tggatccctt tcttttcgga tccgggacgg gagggggagg catatttttt tattatttt |
| SEQ ID NO. 51 | gcctccccct cccgtccc |
| SEQ ID NO. 52 | gggacgggag ggggaggcat atttttttat tatttt |
| SEQ ID NO. 53 | gatccgggac gggaggggga ggc |
| SEQ ID NO. 54 | gggacgggag ggggaggcat atttttttat tatttt |
| SEQ ID NO. 55 | tggatccctt tcttttcgga tccacaatag ggcgaaacat atagatttat tatttt |
| SEQ ID NO. 56 | catatagatt tattatttt |
| SEQ ID NO. 57 | aaaataataa atctatatg |
| SEQ ID NO. 58 | gtttcgccct attgtggatc |
| SEQ ID NO. 59 | nnnnnnnnnn nnnnnnnnnn tttcgcccta ttgt |
| SEQ ID NO. 60 | tggatccctt tcttttcgga tccacaatag ggcgaaannn nnnnnnnnnn nnnnnnn |
| SEQ ID NO. 61 | tttcgcccta ttgt |
| SEQ ID NO. 62 | acaatagggc gaaannnnnn nnnnnnnnnn nnnn |
| SEQ ID NO. 63 | gccggaaaac ctcgatgtg |
| SEQ ID NO. 64 | aaagtaaaaa ctctatatg |
| SEQ ID NO. 65 | aaaataataa atctatatg |
| SEQ ID NO. 66 | aaaataataa atttatat |
| SEQ ID NO. 67 | gcctcgccct cccgt |
| SEQ ID NO. 68 | gcctcgccct cccgtccc |
| SEQ ID NO. 69 | atattttttt attatttt |
| SEQ ID NO. 70 | gggacgggag ggggaggc |
| SEQ ID NO. 71 | tggatccctt tcttttcgga tccgggacgg gagggggagg catatttttt tattatttt |
| SEQ ID NO. 72 | tttcgcccta ttgtggatcc |
| SEQ ID NO. 73 | tttcgcccta ttgt |
| SEQ ID NO. 74 | tggatccctt tcttttcgga tccacaatag ggcgaaa |
| SEQ ID NO. 75 | acaatagggc gaaa |
| SEQ ID NO. 76 | ctgtttcgcc ctattgtgga tcc |
| SEQ ID NO. 77 | cagtgccgca ctattgt |
| SEQ ID NO. 78 | tggatccctt tcttttcgga tccacaatag ggcgaaacag |
| SEQ ID NO. 79 | tggatccctt tcttttcgga tccacaatag ggcgaaa |
| SEQ ID NO. 80 | tggatccctt tttttcgga tccacaatag ggcgaaacag |
| SEQ ID NO. 81 | ggatccctta cttttaggat ccaaaatag |
| SEQ ID NO. 82 | ggatccctta cttttaggat ccacaatagt gcg |
| SEQ ID NO. 83 | cgcactattg t |
| SEQ ID NO. 84 | acaatagtgc g |
| SEQ ID NO. 85 | cgcactattg tggatg |
| SEQ ID NO. 86 | cgcactattg t |
| SEQ ID NO. 87 | tggatccctt acttttagga tccacaatag tgcg |
| SEQ ID NO. 88 | cgcactattg tggatg |
| SEQ ID NO. 89 | cgcactattg t |
| SEQ ID NO. 90 | tggatccctt acttttagga tccacaatag tgcg |

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.
1. A nucleic acid strand displacement system comprising:
   a nucleic acid molecule comprising:
   a toehold region;
   an endonuclease recognition cleavage site; and
   a toehold blocker region configured to couple to the toehold region, wherein the toehold blocker region is further configured to release from the toehold region when a cleavage occurs at the endonuclease recognition cleavage site.
2. The nucleic acid strand displacement system of Embodiment 1, wherein the toehold blocker region is at least partially complementary to the toehold region.
3. The nucleic acid strand displacement system of Embodiment 1, further comprising a restriction endonuclease configured to cleave the endonuclease recognition cleavage site.
4. The nucleic acid strand displacement system of Embodiment 1 or 2, further comprising a signal nucleic acid molecule configured to couple to a signal complementary region of the nucleic acid molecule.
5. The nucleic acid strand displacement system of Embodiment 4, wherein the signal nucleic acid molecule further comprises a fluorophore tag configured to generate a detectable signal.
6. The nucleic acid strand displacement system of Embodiment 5, wherein the nucleic acid molecule further comprises a quencher tag configured to quench the detectable signal when the nucleic acid molecule is coupled to the signal nucleic acid molecule.
7. The nucleic acid strand displacement system of Embodiment 4, further comprising an invading nucleic acid molecule configured to couple to the toehold region and the signal complementary region.
8. The nucleic acid strand displacement system of any of Embodiments 1-7, wherein the nucleic acid molecule further comprises a loop region connecting the toehold region and the toehold blocker region.
9. The nucleic acid strand displacement system of Embodiment 8, wherein the loop region comprises the endonuclease recognition cleavage site.
10. The nucleic acid strand displacement system of any of Embodiments 1-9, wherein the nucleic acid molecule is a first nucleic acid molecule, the toehold region is a first toehold region, the endonuclease recognition cleavage site is a first endonuclease recognition site, the toehold blocker region is a first blocker region, and
   wherein the nucleic acid strand displacement system further comprises a second nucleic acid molecule comprising:
   a second toehold region;
   a second endonuclease recognition cleavage site; and
   a second toehold blocker region configured to couple to the second toehold region, wherein the second toehold blocker region is further configured to release from the second toehold region when a second cleavage occurs at the second endonuclease recognition cleavage site.
11. The nucleic acid strand displacement system of Embodiment 10, wherein the first endonuclease recognition cleavage site is different than the second endonuclease recognition cleavage site.
12. The nucleic acid strand displacement system of any of Embodiments 10 or 11, wherein the first endonuclease recognition cleavage site is configured to be cleaved by a first restriction endonuclease, and wherein the second endonuclease recognition cleavage site is configured to be cleaved by a second restriction endonuclease different than the first restriction endonuclease.
13. The nucleic acid strand displacement system of any of Embodiments 10-12, wherein the nucleic acid strand displacement system further comprises:
   a first signal complementary region configured to couple to the first signal nucleic acid molecule, wherein the first signal nucleic acid molecule comprises a first fluorophore tag configured to generate a detectable signal;
   a first invading nucleic acid molecule configured to couple to the first toehold region and the first signal complementary region;
   a second signal complementary region of the second nucleic acid molecule;
   a second signal nucleic acid molecule configured to couple to the second signal complementary region;
   a second invading nucleic acid molecule configured to couple to the second toehold region and the second signal complementary region;
   a second fluorophore tag coupled to the second signal nucleic acid molecule.
14. The nucleic acid strand displacement system of Embodiment 13, wherein the first fluorophore tag and the second fluorophore tag are configured to generate different detectable signals.
15. A method for detecting the presence or absence of cleavage of an endonuclease cleavage site, the method comprising:
   adding a bacteria sample to a mixture comprising:
   a nucleic acid molecule, wherein the nucleic acid molecule comprises:
   a toehold region;
   an endonuclease recognition cleavage site; and
   a toehold blocker region configured to couple to the toehold region, wherein the toehold blocker region is further configured to release from the toehold region when a restriction endonuclease cleaves the endonuclease recognition cleavage site;
   incubating the mixture under conditions and for a time sufficient to cleave the endonuclease recognition cleavage site in the presence of the restriction endonuclease; and
   detecting, in the mixture, the presence or absence of detectable signal associated with cleavage of the endonuclease recognition cleavage site.
16. The method of Embodiment 15, wherein the nucleic acid molecule further comprises a loop region connecting the toehold region and the toehold blocker region, and wherein the loop region comprises the endonuclease recognition cleavage site.
17. The method of any of Embodiments 15 or 16, wherein the mixture further comprises a signal nucleic acid molecule configured to couple to a signal complementary region of the nucleic acid molecule, and wherein the signal nucleic acid molecule further comprises a fluorophore tag configured to generate a fluorescent detectable signal.
18. The method of Embodiment 17, wherein the nucleic acid molecule further comprises a quencher tag configured to quench fluorescence of the fluorophore tag.
19. The method of any of Embodiments 15-18, wherein the mixture further comprises an invading nucleic acid molecule configured to couple to the toehold region and the signal complementary region.
20. The method of any of Embodiments 15-19, wherein detecting comprises detecting the presence or absence of the detectable signal, wherein the presence of the first detectable signal is indicative of the presence of the restriction endonuclease in the mixture.

1. A nucleic acid strand displacement system comprising:
a nucleic acid molecule comprising:
a toehold region;
an endonuclease recognition cleavage site; and
a toehold blocker region configured to couple to the toehold region, wherein the toehold blocker region is further configured to release from the toehold region when a cleavage occurs at the endonuclease recognition cleavage site.

2. The nucleic acid strand displacement system of claim 1, wherein the toehold blocker region is at least partially complementary to the toehold region.

3. The nucleic acid strand displacement system of claim 1, further comprising a restriction endonuclease configured to cleave the endonuclease recognition cleavage site.

4. The nucleic acid strand displacement system of claim 1, further comprising a signal nucleic acid molecule configured to couple to a signal complementary region of the nucleic acid molecule.

5. The nucleic acid strand displacement system of claim 4, wherein the signal nucleic acid molecule further comprises a fluorophore tag configured to generate a detectable signal.

6. The nucleic acid strand displacement system of claim 5, wherein the nucleic acid molecule further comprises a quencher tag configured to quench the detectable signal when the nucleic acid molecule is coupled to the signal nucleic acid molecule.

7. The nucleic acid strand displacement system of claim 4, further comprising an invading nucleic acid molecule configured to couple to the toehold region and the signal complementary region.

8. The nucleic acid strand displacement system of claim 1, wherein the nucleic acid molecule further comprises a loop region connecting the toehold region and the toehold blocker region.

9. The nucleic acid strand displacement system of claim 8, wherein the loop region comprises the endonuclease recognition cleavage site.

10. The nucleic acid strand displacement system of claim 1, wherein the nucleic acid molecule is a first nucleic acid molecule, the toehold region is a first toehold region, the endonuclease recognition cleavage site is a first endonuclease recognition site, the toehold blocker region is a first blocker region, and
wherein the nucleic acid strand displacement system further comprises a second nucleic acid molecule comprising:
a second toehold region;
a second endonuclease recognition cleavage site; and
a second toehold blocker region configured to couple to the second toehold region, wherein the second toehold blocker region is further configured to release from the second toehold region when a second cleavage occurs at the second endonuclease recognition cleavage site.

11. The nucleic acid strand displacement system of claim 10, wherein the first endonuclease recognition cleavage site is different than the second endonuclease recognition cleavage site.

12. The nucleic acid strand displacement system of claim 10, wherein the first endonuclease recognition cleavage site is configured to be cleaved by a first restriction endonuclease, and wherein the second endonuclease recognition cleavage site is configured to be cleaved by a second restriction endonuclease different than the first restriction endonuclease.

13. The nucleic acid strand displacement system of claim 10, wherein the nucleic acid strand displacement system further comprises:
a first signal complementary region configured to couple to the first signal nucleic acid molecule, wherein the first signal nucleic acid molecule comprises a first fluorophore tag configured to generate a detectable signal;
a first invading nucleic acid molecule configured to couple to the first toehold region and the first signal complementary region;
a second signal complementary region of the second nucleic acid molecule;
a second signal nucleic acid molecule configured to couple to the second signal complementary region;
a second invading nucleic acid molecule configured to couple to the second toehold region and the second signal complementary region;
a second fluorophore tag coupled to the second signal nucleic acid molecule.

14. The nucleic acid strand displacement system of claim 13, wherein the first fluorophore tag and the second fluorophore tag are configured to generate different detectable signals.

15. A method for detecting the presence or absence of cleavage of an endonuclease cleavage site, the method comprising:
adding a bacteria sample to a mixture comprising:
a nucleic acid molecule, wherein the nucleic acid molecule comprises:
a toehold region;
an endonuclease recognition cleavage site; and
a toehold blocker region configured to couple to the toehold region, wherein the toehold blocker region is further configured to release from the toehold region when a restriction endonuclease cleaves the endonuclease recognition cleavage site;
incubating the mixture under conditions and for a time sufficient to cleave the endonuclease recognition cleavage site in the presence of the restriction endonuclease; and
detecting, in the mixture, the presence or absence of detectable signal associated with cleavage of the endonuclease recognition cleavage site.

16. The method of claim 15, wherein the nucleic acid molecule further comprises a loop region connecting the toehold region and the toehold blocker region, and wherein the loop region comprises the endonuclease recognition cleavage site.

17. The method of claim 15, wherein the mixture further comprises a signal nucleic acid molecule configured to couple to a signal complementary region of the nucleic acid molecule, and wherein the signal nucleic acid molecule further comprises a fluorophore tag configured to generate a fluorescent detectable signal.

18. The method of claim 17, wherein the nucleic acid molecule further comprises a quencher tag configured to quench fluorescence of the fluorophore tag.

19. The method of claim 15, wherein the mixture further comprises an invading nucleic acid molecule configured to couple to the toehold region and the signal complementary region.

20. The method of claim 15, wherein detecting comprises detecting the presence or absence of the detectable signal, wherein the presence of the first detectable signal is indicative of the presence of the restriction endonuclease in the mixture.


SEQUENCE LISTING (90 sequences)

SEQ ID NO: 1 - DNA, 32 bp, synthetic construct
  source 1..32: mol_type = other DNA
ggatcccttactttaggatccacaatagtgcg

SEQ ID NO: 2 - DNA, 35 bp, synthetic construct
  source 1..35: mol_type = other DNA
gtggatcccttactttccggatccacaatactccg

SEQ ID NO: 3 - DNA, 44 bp, synthetic construct
  source 1..44: mol_type = other DNA
tttggatcccccaaacaaaccggggatccaaatttataccgccg

SEQ ID NO: 4 - DNA, 44 bp, synthetic construct; identical to SEQ ID NO: 3
  source 1..44: mol_type = other DNA
  misc_feature 25..32: note = toehold region (b')
  misc_feature 1..8: note = toehold blocker region (b)
  misc_feature 24..29: note = restriction endonuclease (NA)
  misc_feature 33..44: note = signal complementary region (a')
  misc_feature 10..24: note = loop region (c)

SEQ ID NO: 5 - DNA, 12 bp, synthetic construct
  source 1..12: mol_type = other DNA
cggcggtataaa

SEQ ID NO: 6 - DNA, 15 bp, synthetic construct
  source 1..15: mol_type = other DNA
cggcggtataaattt

SEQ ID NO: 7 - DNA, 20 bp, synthetic construct
  source 1..20: mol_type = other DNA
cggcggtataaatttggatc

SEQ ID NO: 8 - DNA, 20 bp, synthetic construct
  source 1..20: mol_type = other DNA
cggcggtataaatttggatc

SEQ ID NO: 9 - DNA, 15 bp, synthetic construct
  source 1..15: mol_type = other DNA
cggcggtataaattt

SEQ ID NO: 10 - DNA, 44 bp, synthetic construct; identical to SEQ ID NO: 3
  source 1..44: mol_type = other DNA

SEQ ID NO: 11 - DNA, 50 bp, synthetic construct
  source 1..50: mol_type = other DNA
atttggatccgcccggaaacaacccgggcggatccaaatttataccgccg

SEQ ID NO: 12 - DNA, 40 bp, synthetic construct
  source 1..40: mol_type = other DNA
tttggatcccttactttccggatccaaagaaaaaccgccg

SEQ ID NO: 13 - DNA, 15 bp, synthetic construct
  source 1..15: mol_type = other DNA
cggcggtttttcttt

SEQ ID NO: 14 - DNA, 20 bp, synthetic construct
  source 1..20: mol_type = other DNA
cggcggtttttctttggatc

SEQ ID NO: 15 - DNA, 50 bp, synthetic construct; identical to SEQ ID NO: 11
  source 1..50: mol_type = other DNA

SEQ ID NO: 16 - DNA, 50 bp, synthetic construct
  source 1..50: mol_type = other DNA
cggcggtataaatttggatccgcccgggttgtttccgggcggatccaaat

SEQ ID NO: 17 - DNA, 57 bp, synthetic construct
  source 1..57: mol_type = other DNA
atttggatccgcccggcccaaacaaaccggccgggcggatccaaatttataccgccg

SEQ ID NO: 18 - DNA, 57 bp, synthetic construct
  source 1..57: mol_type = other DNA
cggcggtataaatttggatccgcccggccggtttgtttgggccgggcggatccaaat

SEQ ID NO: 19 - DNA, 50 bp, synthetic construct
  source 1..50: mol_type = other DNA
tttggatccccccccaaacaaaccgggggggatccaaatttataccgccg

SEQ ID NO: 20 - DNA, 44 bp, synthetic construct; identical to SEQ ID NO: 3
  source 1..44: mol_type = other DNA

SEQ ID NO: 21 - DNA, 20 bp, synthetic construct
  source 1..20: mol_type = other DNA
cggcggtataaatttggatc

SEQ ID NO: 22 - DNA, 15 bp, synthetic construct
  source 1..15: mol_type = other DNA
cggcggtataaattt

SEQ ID NO: 23 - DNA, 40 bp, synthetic construct; identical to SEQ ID NO: 12
  source 1..40: mol_type = other DNA

SEQ ID NO: 24 - DNA, 20 bp, synthetic construct
  source 1..20: mol_type = other DNA
cggcggtttttctttggatc

SEQ ID NO: 25 - DNA, 15 bp, synthetic construct
  source 1..15: mol_type = other DNA
cggcggtttttcttt

SEQ ID NO: 26 - DNA, 40 bp, synthetic construct; identical to SEQ ID NO: 12
  source 1..40: mol_type = other DNA

SEQ ID NO: 27 - DNA, 20 bp, synthetic construct
  source 1..20: mol_type = other DNA
cggcggtttttctttggatc

SEQ ID NO: 28 - DNA, 15 bp, synthetic construct
  source 1..15: mol_type = other DNA
cggcggtttttcttt

SEQ ID NO: 29 - DNA, 40 bp, synthetic construct; identical to SEQ ID NO: 12
  source 1..40: mol_type = other DNA

SEQ ID NO: 30 - DNA, 40 bp, synthetic construct
  source 1..40: mol_type = other DNA
cggcggtttttctttggatccggaaagtaagggatccaaa

SEQ ID NO: 31 - DNA, 35 bp, synthetic construct
  source 1..35: mol_type = other DNA
gtggatcccttactttccggatccacaaaccgccg

SEQ ID NO: 32 - DNA, 16 bp, synthetic construct
  source 1..16: mol_type = other DNA
cggcggtttgtggatc

SEQ ID NO: 33 - DNA, 11 bp, synthetic construct
  source 1..11: mol_type = other DNA
cggcggtttgt

SEQ ID NO: 34 - DNA, 35 bp, synthetic construct; identical to SEQ ID NO: 31
  source 1..35: mol_type = other DNA

SEQ ID NO: 35 - DNA, 35 bp, synthetic construct
  source 1..35: mol_type = other DNA
cggcggtttgtggatccggaaagtaagggatccac

SEQ ID NO: 36 - DNA, 35 bp, synthetic construct; identical to SEQ ID NO: 2
  source 1..35: mol_type = other DNA

SEQ ID NO: 37 - DNA, 16 bp, synthetic construct
  source 1..16: mol_type = other DNA
cggagtattgtggatc

SEQ ID NO: 38 - DNA, 11 bp, synthetic construct
  source 1..11: mol_type = other DNA
cggagtattgt

SEQ ID NO: 39 - DNA, 35 bp, synthetic construct; identical to SEQ ID NO: 2
  source 1..35: mol_type = other DNA

SEQ ID NO: 40 - DNA, 29 bp, synthetic construct
  source 1..29: mol_type = other DNA
gtcccctccccctcccgtccctccggatc

SEQ ID NO: 41 - DNA, 42 bp, synthetic construct
  source 1..42: mol_type = other DNA
aaaataataaaaaaatatgtcccctccccctcccgtccctcc

SEQ ID NO: 42 - DNA, 65 bp, synthetic construct
  source 1..65: mol_type = other DNA
tggatccctttcttttcggatccggagggacgggagggggaggggacatatttttttattatttt

SEQ ID NO: 43 - DNA, 18 bp, synthetic construct
  source 1..18: mol_type = other DNA
gcctccccctcccgtccc

SEQ ID NO: 44 - DNA, 42 bp, synthetic construct
  source 1..42: mol_type = other DNA
ggagggacgggagggggaggggacatatttttttattatttt

SEQ ID NO: 45 - DNA, 29 bp, synthetic construct
  source 1..29: mol_type = other DNA
gatccggagggacgggagggggaggggac

SEQ ID NO: 46 - DNA, 42 bp, synthetic construct; identical to SEQ ID NO: 44
  source 1..42: mol_type = other DNA

SEQ ID NO: 47 - DNA, 59 bp, synthetic construct
  source 1..59: mol_type = other DNA
tggatccctttcttttcggatccgggacgggagggggaggcatatttttttattatttt

SEQ ID NO: 48 - DNA, 23 bp, synthetic construct
  source 1..23: mol_type = other DNA
gcctccccctcccgtcccggatc

SEQ ID NO: 49 - DNA, 36 bp, synthetic construct
  source 1..36: mol_type = other DNA
aaaataataaaaaaatatgcctccccctcccgtccc

SEQ ID NO: 50 - DNA, 59 bp, synthetic construct; identical to SEQ ID NO: 47
  source 1..59: mol_type = other DNA

SEQ ID NO: 51 - DNA, 18 bp, synthetic construct
  source 1..18: mol_type = other DNA
gcctccccctcccgtccc

SEQ ID NO: 52 - DNA, 36 bp, synthetic construct
  source 1..36: mol_type = other DNA
gggacgggagggggaggcatatttttttattatttt

SEQ ID NO: 53 - DNA, 23 bp, synthetic construct
  source 1..23: mol_type = other DNA
gatccgggacgggagggggaggc

SEQ ID NO: 54 - DNA, 36 bp, synthetic construct; identical to SEQ ID NO: 52
  source 1..36: mol_type = other DNA

SEQ ID NO: 55 - DNA, 56 bp, synthetic construct
  source 1..56: mol_type = other DNA
tggatccctttcttttcggatccacaatagggcgaaacatatagatttattatttt

SEQ ID NO: 56 - DNA, 19 bp, synthetic construct
  source 1..19: mol_type = other DNA
catatagatttattatttt

SEQ ID NO: 57 - DNA, 19 bp, synthetic construct
  source 1..19: mol_type = other DNA
aaaataataaatctatatg

SEQ ID NO: 58 - DNA, 20 bp, synthetic construct
  source 1..20: mol_type = other DNA
gtttcgccctattgtggatc

SEQ ID NO: 59 - DNA, 34 bp, synthetic construct
  source 1..34: mol_type = other DNA
nnnnnnnnnnnnnnnnnnnntttcgccctattgt

SEQ ID NO: 60 - DNA, 57 bp, synthetic construct
  source 1..57: mol_type = other DNA
tggatccctttcttttcggatccacaatagggcgaaannnnnnnnnnnnnnnnnnnn

SEQ ID NO: 61 - DNA, 14 bp, synthetic construct
  source 1..14: mol_type = other DNA
tttcgccctattgt

SEQ ID NO: 62 - DNA, 34 bp, synthetic construct
  source 1..34: mol_type = other DNA
acaatagggcgaaannnnnnnnnnnnnnnnnnnn

SEQ ID NO: 63 - DNA, 19 bp, synthetic construct
  source 1..19: mol_type = other DNA
gccggaaaacctcgatgtg

SEQ ID NO: 64 - DNA, 19 bp, synthetic construct
  source 1..19: mol_type = other DNA
aaagtaaaaactctatatg

SEQ ID NO: 65 - DNA, 19 bp, synthetic construct
  source 1..19: mol_type = other DNA
aaaataataaatctatatg

SEQ ID NO: 66 - DNA, 18 bp, synthetic construct
  source 1..18: mol_type = other DNA
aaaataataaatttatat

SEQ ID NO: 67 - DNA, 15 bp, synthetic construct
  source 1..15: mol_type = other DNA
gcctcgccctcccgt

SEQ ID NO: 68 - DNA, 18 bp, synthetic construct
  source 1..18: mol_type = other DNA
gcctcgccctcccgtccc

SEQ ID NO: 69 - DNA, 18 bp, synthetic construct
  source 1..18: mol_type = other DNA
atatttttttattatttt

SEQ ID NO: 70 - DNA, 18 bp, synthetic construct
  source 1..18: mol_type = other DNA
gggacgggagggggaggc

SEQ ID NO: 71 - DNA, 59 bp, synthetic construct; identical to SEQ ID NO: 47
  source 1..59: mol_type = other DNA

SEQ ID NO: 72 - DNA, 20 bp, synthetic construct
  source 1..20: mol_type = other DNA
tttcgccctattgtggatcc

SEQ ID NO: 73 - DNA, 14 bp, synthetic construct
  source 1..14: mol_type = other DNA
tttcgccctattgt

SEQ ID NO: 74 - DNA, 37 bp, synthetic construct
  source 1..37: mol_type = other DNA
tggatccctttcttttcggatccacaatagggcgaaa

SEQ ID NO: 75 - DNA, 14 bp, synthetic construct
  source 1..14: mol_type = other DNA
acaatagggcgaaa

SEQ ID NO: 76 - DNA, 23 bp, synthetic construct
  source 1..23: mol_type = other DNA
ctgtttcgccctattgtggatcc

SEQ ID NO: 77 - DNA, 17 bp, synthetic construct
  source 1..17: mol_type = other DNA
cagtgccgcactattgt

SEQ ID NO: 78 - DNA, 40 bp, synthetic construct
  source 1..40: mol_type = other DNA
tggatccctttcttttcggatccacaatagggcgaaacag

SEQ ID NO: 79 - DNA, 37 bp, synthetic construct; identical to SEQ ID NO: 74
  source 1..37: mol_type = other DNA

SEQ ID NO: 80 - DNA, 40 bp, synthetic construct; identical to SEQ ID NO: 78
  source 1..40: mol_type = other DNA

SEQ ID NO: 81 - DNA, 29 bp, synthetic construct
  source 1..29: mol_type = other DNA
ggatcccttacttttaggatccaaaatag

SEQ ID NO: 82 - DNA, 33 bp, synthetic construct
  source 1..33: mol_type = other DNA
ggatcccttacttttaggatccacaatagtgcg

SEQ ID NO: 83 - DNA, 11 bp, synthetic construct
  source 1..11: mol_type = other DNA
cgcactattgt

SEQ ID NO: 84 - DNA, 11 bp, synthetic construct
  source 1..11: mol_type = other DNA
acaatagtgcg

SEQ ID NO: 85 - DNA, 16 bp, synthetic construct
  source 1..16: mol_type = other DNA
cgcactattgtggatg

SEQ ID NO: 86 - DNA, 11 bp, synthetic construct
  source 1..11: mol_type = other DNA
cgcactattgt

SEQ ID NO: 87 - DNA, 34 bp, synthetic construct
  source 1..34: mol_type = other DNA
tggatcccttacttttaggatccacaatagtgcg

SEQ ID NO: 88 - DNA, 16 bp, synthetic construct
  source 1..16: mol_type = other DNA
cgcactattgtggatg

SEQ ID NO: 89 - DNA, 11 bp, synthetic construct
  source 1..11: mol_type = other DNA
cgcactattgt

SEQ ID NO: 90 - DNA, 34 bp, synthetic construct; identical to SEQ ID NO: 87
  source 1..34: mol_type = other DNA